(19) United States
(12) Patent Application Publication
(10) Pub. No.: US 2026/0175372 A1
(43) Pub. Date: Jun. 25, 2026

(54) DEVICE FOR DOSING AND DISPENSING SOLID ELEMENTS SUCH AS MEDICINE TABLETS

(71) Applicant: Navamedic AB, Sävedalen (SE)

(72) Inventors: Jack SPIRA, Tyresö (SE); Rasmus ERIKSSON, Hägersten (SE)

(73) Assignee: Navamedic AB, Sävedalen (SE)

(21) Appl. No.: 19/124,508

(22) PCT Filed: Oct. 26, 2023
(86) PCT No.: PCT/SE2023/051054

§ 371 Date: Apr. 25, 2025

(30) Foreign Application Priority Data
Oct. 28, 2022 (SE) 2251250-3

(51) Int. Cl. B25B 13/46; A61M 15/00; B65D 83/04

(52) U.S. Cl. CPC B25B 13/463; A61M 15/009; B25B 13/465; B65D 83/0409

(57) ABSTRACT

A device for dosing and dispensing solid elements includes: a feeding unit; a separating unit; and a dispensing unit; wherein: the feeding unit, separating unit and dispensing unit are arranged to rotate relative each other. The separating unit includes a plurality of recesses or through holes, each having a size such that the recess or through hole is capable of containing a predetermined number of solid elements. A compartment for containing the solid elements is formed at least partly by the feeding unit, and the feeding unit includes at least one feeding opening that opens towards the separating unit to feed solid elements from the compartment to one or more of the recesses or through holes that are rotationally aligned with the at least one feeding opening.

### TECHNICAL FIELD

The invention relates to the field of devices for dosing and dispensing solid elements such as tablets comprising medicine for the treatment or prevention of a disease and/or nutritional supplements.

### BACKGROUND

The treatment, control, or prevention of certain diseases such as Parkinson's disease have a narrow therapeutic window and thus require accurate dosing to provide the desired effect and to avoid side effects. Other examples of such diseases include, but is not limited to, epilepsy, cancer, depression, ADHD, schizophrenia. For some nutritional supplements, accurate dosing is also important.

Devices for dosing and dispensing, i.e. counting a number of tablets and ejecting the tablets to the user, are known in the art. For example, in the applicant's earlier patents EP 2367519 B1 and EP 2948390 B1, an electronic device comprising a controller and drive motors is disclosed, which device doses and dispenses tablets contained in cassettes.

Non-motorized devices are also known in the art. For example, EP 2847096 B1, discloses a device comprising a metering surface provided with depressions in which tablets can be retained. U.S. Pat. No. 9,815,611 B2 discloses a different type of device in which the user rotates a wheel comprising chambers on its inner side in which tablets are received and transferred to a receiver provided with a dosing scale.

Although the non-motorized devices described above are advantageous in terms of cost and complexity, the dosing accuracy may be unsatisfactory since the number of tablets must be visually determined; in EP 2847096 B1 the tablets on the metering surface are manually counted, and in U.S. Pat. No. 9,815,611 B2, the number of tablets is visually determined using the dosing scale. Such visual determination may be challenging for some users, Parkinson's disease patients in particular.

### SUMMARY

An object of the invention is to provide a manual/non-motorized dosing and dispensing device which provides accurate dosing and ease of use.

These and other objects are achieved by the present invention by means of a device according to the independent claim.

According to a first aspect of the invention, there is provided a device for dosing and/or dispensing solid elements such as medicine tablets, comprising a feeding unit, a separating unit and a dispensing unit. The feeding unit, separating unit and dispensing unit are arranged to rotate relative each other. The separating unit comprises a plurality of recesses or through holes. Each recess or through hole may have a size adapted to contain a predetermined number of solid elements, such as one solid element A compartment for containing said solid elements is formed at least partly by said feeding unit, and wherein said feeding unit is provided with at least one feeding opening towards the separating unit to feed solid elements from said compartment to each of one or more of said recesses or through holes which are rotationally aligned with said at least one feeding opening. The dispensing unit comprises a guiding surface arranged to co-act with the separating unit, the guiding surface comprising a retaining portion arranged to form a wall portion (such as a lower wall portion) for one or more recesses or through holes of the separating unit which are rotationally aligned with said retaining portion, and a dispensing portion comprising a dispensing opening arranged to allow solid elements contained in one or more recesses or through holes of the separating unit to be dispensed. A first one-way rotation mechanism is configured to allow rotation of the feeding unit relative the separating unit solely in a first direction of rotation. The first one-way rotation mechanism comprises, or is arranged to co-act with, stepping means subdividing said rotation in the first direction into angular steps which correspond to an angular distance between a predetermined number of said recesses or through holes. A second one-way rotation mechanism is configured to allow rotational movement of the separating unit relative the dispensing unit solely in a second direction of rotation being opposite the first direction of rotation.

In other words, the device comprises three moveable parts, a feeding unit, a separating unit and a dispensing unit, which are rotatable relative each other, preferably around a common axis of rotation. The separating unit is arranged between the feeding unit and the dispensing unit and may be described as sandwiched between the feeding unit and the dispensing unit. The feeding unit and the dispensing unit may be rotatable relative each other between an initial position and an end position such that the dispensing opening and the feeding opening do not overlap. Put differently, the plurality of recesses or through holes of said separating unit can be said to form chambers for one or more tablets. The separating unit may have a substantially circular cross-section, and the plurality of recesses or through holes may be formed at or adjacent the periphery of the separating unit, i.e. in a circular formation. Since the separating unit is rotatable, the chambers/recesses/through holes are rotatable along a movement path, such as a circular movement path. The compartment for containing the solid elements is formed at least partly by said feeding unit in the sense that the compartment may be formed in the feeding unit alone or may be formed by the feeding unit along with another part. The solid elements may be medicine tablets or medicine granulates, i.e. the device may be adapted/configured for dosing and dispensing medicine tablets or medicine granulates. When the device is used with/adapted for medicine granulates, each recess or through hole of the separating unit typically contains more than one granulate (solid element). The device may be adapted/configured for dosing and dispensing medicine tablets in the form of minitablets having a size within a range of 1-6 mm such as 2-5 mm or 3-4 mm.

The at least one feeding opening towards the separating unit is formed such as to open towards one or more recess or through hole which is/are rotationally aligned with the at least one feeding opening. The guiding surface of the dispensing unit is arranged to co-act with the separating unit for example by being arranged substantially in abutment with the separating unit, i.e. such that the separating rotates on (top of) the guiding surface or at a short distance therefrom. The retaining portion of the guiding surface is arranged to form a wall portion (being a lower wall portion when the dispensing unit is arranged below the separating unit) for one or more recesses or through holes of the separating unit which are rotationally aligned with said retaining portion to prevent solid elements contained therein to leave. The guiding surface further comprises a dispensing portion comprising a dispensing opening arranged to allow solid elements contained in one or more recesses or through holes of the separating unit which are rotationally aligned with the dispensing opening to be dispensed.

The function of the first and second one-way rotation mechanisms can be described as follows. In the first direction of rotation, the feeding unit can be rotated relative the separating unit (due to the first one-way rotation mechanism) and relative dispensing unit (neither the first nor the second one-way rotation mechanism engage between the feeding unit and the dispensing unit). In the second direction of rotation, the feeding unit rotates together with the separating unit (due to the first one-way rotation mechanism) relative the dispensing unit (due to the second one-way rotation mechanism).

The feeding opening may be described as arranged ahead of the dispensing opening as seen in the first direction of rotation such that when the feeding unit is rotated relative the separating unit and dispensing unit in the first direction, solid elements are fed to one or more of the recesses or through holes in the separating unit (but is prevented from leaving due to the retaining portion of the guiding surface), and such that when the feeding unit is rotated relative the dispensing unit in the second direction, solid elements are dispensed through the dispensing opening.

The stepping means provides the function that each angular step which the feeding unit is rotated corresponds to a certain number of solid elements being filled into the through holes or recesses of the separating unit. The number is equal to the predetermined number of recesses or through holes which the angular distance corresponds to (preferably one) times the predetermined number of solid elements which each recess or through holes is adapted to contain (preferably one). Consequently, each angular step which the feeding unit is rotated increases the dose with a certain number of solid elements (preferably one). Consequently, the dosing can conveniently be determined by the number of steps rotated. The stepping means may be formed by (or together with) the first one-way rotation mechanism or may be a separate stepping mechanism.

In embodiments, the device may be provided with a counting indicator indicating the number of angular steps rotated or the total number of solid elements filled into the through holes/recesses of the separating unit (which is equal to the predetermined number of recesses or through holes which the angular distance corresponds to (such as one) times the predetermined number of solid elements which each recess or through holes is adapted to contain (such as one)). For example, the dispensing unit may be provided with a numbered collar, and the feeding unit may be provided with a ridge portion which is rotationally displaced along the numbered collar to indicate the number of steps or number of solid elements.

According to a second aspect of the invention, there is provided an arrangement comprising a device according to the first aspect of the invention or embodiments thereof and a plurality of solid elements being tablets contained in the compartment of said device. The tablets may comprise medicine for the treatment or prevention of a disease and/or nutritional supplements.

The invention is based on the insight that by separating counting/dosing from dispensing into two separate rotational movements in opposite directions, the through holes/recesses can be accurately filled with solid elements during the first rotational movement such that the number of solid elements can be counted by means of stepping means subdividing the first rotational movement into angular steps which correspond to an angular distance between a predetermined number of recesses or through holes. Consequently, the number of solid elements accurately correlates with the number of steps of rotation, and visual determination of the number of tablets is not necessary.

In embodiments, the first one-way rotation mechanism and the stepping means are formed together as a ratchet mechanism comprising a first ratchet gear portion of said separating unit and at least one first ratchet finger of said feeding unit engaging with each other. The first ratchet gear portion may be formed as an outer gear on a portion of the separating unit, i.e. a toothed profile at the periphery of a portion of the separating unit. The ratchet finger(s) may also be referred to as a pawl(s). Such embodiments are advantageous since the number of parts is kept low (since the two mechanisms are both realized using the same parts).

In embodiments, the second one-way rotation mechanism is formed as a ratchet mechanism comprising a second ratchet gear portion of said separating unit and at least one second ratchet finger of said dispensing unit engaging with each other. The second ratchet gear portion may be formed as an inner gear on the separating unit, i.e. a toothed profile at an inner periphery of the separating unit. The ratchet finger(s) may also be referred to as a pawl(s). The steps of the second ratchet gear portion may be half as long as the steps of first ratchet gear portion.

In other embodiments, the first and/or second one-way rotation mechanisms is/are formed by other means than ratchet mechanisms, for example, the first and/or second one-way rotation mechanisms may comprise sprag clutch(es). In embodiments where the first one-way rotation mechanism comprises a sprag clutch, the stepping means needs to be formed separately.

In embodiments, the through holes of the separating unit each have a size, diameter or cross-section corresponding to (n+d) times a size or diameter of one of the solid elements, where n is an integer >=1, and d is within an interval from 0.1 to 0.7. For example, the size of the through holes can be 1.1-1.7 times the diameter of the solid elements (n=1). Such embodiments are advantageous since such a size allows one solid element to fit easily, but since the size is sufficiently far from twice the diameter, there is no risk that two solid elements can get wedged in the through holes.

In embodiments, the first ratchet gear of the separating unit is formed with ratchet teeth having a leading side comprising first and second portions, wherein the first portion is arranged radially inwards from the second portion, and wherein the first portion has a steeper inclination than the second portion. This may be advantageous since the first portion (with steep inclination) results in that a high force of rotation is required during initial rotational movement such that the user of the device does not rotate the feeding unit by mistake.

In embodiments, the dispensing unit further comprises a dispensing chamber arranged in communication with the dispensing opening such that the solid elements are dispensed into the dispensing chamber. The chamber of the dispensing unit may be provided with an outlet opening, and the dispensing unit may be provided with a release unit being displaceable by a user of the device between a closed position and an open position, wherein in the closed position, the release unit blocks the outlet opening to prevent solid elements from leaving the dispensing chamber, and wherein in said open position, an outlet of the release unit overlaps at least partly with the outlet opening to allow solid elements to leave said dispensing chamber. Such embodiments comprising a dispensing chamber are advantageous since during operation, the feeding unit is rotated in the first direction to count the number of tablets, and thereafter during rotation in the second direction, the tablets are dispensed one at a time from the through holes or recesses in the separating unit via the dispensing opening into the dispensing chamber, and thereafter the tablets are ejected all at once via the outlet opening. Thus, the counting (by means of rotation in the first and second directions) and ejecting the tablets (by user displacement of the release unit) are separated, resulting in a device which is convenient to use.

In embodiments, the device further comprises a restricting member arranged to co-act with the feeding unit and the dispensing unit such as to allow the release unit to be displaced to said open position only at a predetermined relative position between the feeding unit and the dispensing unit. The restricting member may be arranged to co-act with the feeding unit and the dispensing unit by means of the restricting member being arranged to rotate with the feeding unit and being provided with a slot, and by the release unit being provided with a protrusion such as to allow the release unit to be displaced to said open position only at a predetermined relative position between the feeding unit and the dispensing unit when the protrusion is aligned with the slot to allow the protrusion to enter therein.

In embodiments, the restricting member may, in addition to the above-described restricting function, or as an alternative to said restricting function, be configured to hold the feeding unit, separating unit and dispensing unit together. For example, the restricting member may be configured to engage with a portion of the feeding unit protruding axially through the separating unit and dispensing unit. In such embodiments, the restricting member is important for holding the device together. The restricting member may be manufactured from polyoxymethylene (POM). Based on drop tests, POM has proven to provide advantageous durability of the device.

The restricting member may be provided with snap elements engaging with (through holes or recesses in) said portion of the feeding unit By optimizing the snap elements to reduce the tension therein when engaged with the feeding unit, it is foreseeable that the restricting member may be manufactured from other materials such as polypropylene (PP).

In embodiments, the at least one feeding opening is provided as a feeding opening having an angular extension corresponding to the angular extension of said one or more of said recesses or through holes. The angular extension may correspond to the angular extension of two to ten recesses or through holes, such as four recesses or through holes. A feeding opening having an angular extension corresponding to two or more through holes may be advantageous since each through hole or recess is exposed towards the feeding opening for a longer time. In an embodiment where the stepping means subdivides the rotation in the first direction into angular steps which correspond to one angular distance between one recess or through hole, each through hole or recess is exposed towards the feeding opening during at least two angular steps, which thus provides at least two chances for filling each through hole or recess. This may improve the dosing accuracy.

In embodiments, the feeding device is provided with at least one impacting element arranged in said compartment or forming a wall portion for said compartment (such as a wall portion of said feeding unit), said impacting element being at least partly displaceable such as to displace or agitate one or more solid element stored in said compartment. The impacting element may be displaceable by means of having a free end which is deflectable, for example by means of contact with a gear portion of said separating unit, such as a toothed profile at the periphery of the separating unit. The feeding unit may be provided with at least two impacting elements arranged in parallel. The impacting element(s) displaces/agitates the solid elements being in contact with (leaning/abutting against) the impacting element(s), but since a plurality of solid elements are typically stored in said compartment, the displacement/agitation propagates from the solid elements being in contact with the impacting element(s) to the other solid elements. The impacting element(s) may be arranged substantially perpendicular to the separating unit (in a resting/non-displaced position of the impacting element(s).

In embodiments, the device further comprises a housing part, wherein said compartment is formed by said housing part and said feeding unit.

In embodiments, the device further comprises at least one roof portion or part separating the compartment into an upper and a lower part, said at least one roof portion or part covering part of the cross-section of the feeding unit such as to form at least one opening between said upper and lower part. The roof portion may be a portion of the feeding unit, i.e. integrally formed, or may be a separate part, i.e. a roof part, which for example is arranged in the feeding unit, or in the housing part, or between the feeding unit and the housing part. The at least one roof portion may comprise at least one surface inclining towards said lower part of the compartment One or more of the at least one surface may be provided with at least one raised rib or at least one slot arranged in the at least one surface, the at least one raised rib or slot being arranged at an angle relative its direction of inclination, for instance perpendicularly. In an alternative embodiment, the roof portion is provided with a plurality of through holes. The above-described embodiments comprising at least one roof portion is advantageous since the roof portion(s) reduce the pressure on the tablets near the feeding opening by the column of tablets lying thereon. Such a reduction in pressure allows the tablets to move more easily, which means that the through holes or recesses in the separating unit are more easily filled. This may improve dosing accuracy.

In embodiments comprising at least one inclining surface, the feeding unit may be provided with a tapering portion (which may alternatively be referred to as a funnel portion) tapering towards the feeding opening, wherein said roof portion, at said opening, is arranged at a distance from said tapering portion, the distance being (n+d) times a size or diameter of one of the solid elements, where n is an integer >=1, and d is within an interval from 0.1 to 0.7. For example, the distance can be 1.1-1.7 times the diameter of the solid elements (n=1). Such embodiments are advantageous since the allow one solid element to pass easily, but since the distance is sufficiently far from twice the diameter, there is no risk that two solid elements can get wedged between the roof portion and the tapering portion.

In embodiments where the roof portion is provided with a plurality of through holes, the through holes each have a size, diameter or cross-section corresponding to (n+d) times a size or diameter of one of the solid elements, where n is an integer >=1, and d is within an interval from 0.1 to 0.7. For example, the size of the through holes can be 1.1-1.7 times the diameter of the solid elements (n=1). Such embodiments are advantageous since such a size allows one solid element to pass easily, but since the size is sufficiently far from twice the diameter, there is no risk that two solid elements can get wedged in the through holes.

In embodiments, the device further comprises a stop member being fixable at predetermined positions around the periphery of the feeding unit and/or dispensing unit, the stop member being arranged to co-act with the separating unit and said dispensing unit such as to limit relative rotation therebetween to a number of angular steps corresponding to the predetermined position at which the stop member is fixed. In other words, the relative rotation can be limited to a number of angular steps corresponding to the number of solid elements to be dosed. The stop member may comprise a sleeve portion arranged coaxially with and rotatable relative the feeding unit, an indicating portion being arranged to indicate the predetermined position of the stop member, the stop member further being provided with a limiting member which extends vertically down into the interior of the dispensing unit to act as a stop for a radially outwardly protruding rotation restricting member of the feeding unit such that relative rotation between the separating unit and said dispensing unit is limited to a number of angular steps corresponding to the predetermined position at which the stop member is fixed.

In embodiments, the feeding unit is provided with a feeding surface portion, the feeding surface portion being adjacent to said feeding opening and facing in said first direction of rotation, wherein said feeding surface portion inclines towards said dispensing opening. In embodiments where the feeding unit and said separating unit are arranged to rotate around a common axis of rotation, the feeding surface portion inclines at an angle relative a plane having said common axis of rotation as a normal, said angle being within an interval 100-130 degrees, such as 115 degrees. Such an inclined feeding surface portion is advantageous since the solid elements are pushed in a forwards and upwards direction (in the first direction of rotation and away from the separating unit) which means that rotation of the solid elements is promoted, which in turn allows the through holes or recesses to be filled efficiently without risking crushing the solid elements.

The feeding surface portion may have a width (radial extension defined by said common axis of rotation) being (n+d) times the size or diameter of one of the solid elements, where n is an integer >=1, and d is within an interval from 0.1 to 0.7. Such embodiments are advantageous since it is avoided that solid elements get stuck. For example, the width of the size of the through holes can be 1.1-1.7 times the diameter of the solid elements (n=1). Such a width is sufficient to avoid that one solid element gets stuck, but is sufficiently far from twice the diameter, so there is no risk that two solid elements get wedged.

In embodiments, at least two of the through holes or recesses are each provided with a chamfered or rounded inlet portion towards the feeding unit, wherein peripheries of the openings formed by adjacent inlet portions are in contact with each other. Such a geometry is advantageous since the solid elements easily fall into the through holes or recesses and do not lie on the separating unit without falling into a through hole or recess. In embodiments of the arrangement, the chamfering or rounding of the inlet portion has a height being within an interval of 0.2 to 0.7, such as 0.4 to 0.5, such as 0.42, times a radius of the solid elements. Such a chamfering or rounding is advantageous since rotation of the solid elements is promoted.

In embodiments, at least two of the through holes or recesses are each provided with a chamfered or rounded outlet portion towards the dispensing unit Such a geometry is advantageous since the solid elements easily fall out from the through holes or recesses. In embodiments of the arrangement, the chamfering or rounding of the outlet portion has a height being within an interval of 0.5 to 1.5, such as 0.7 to 1.0, such as 0.8, times a radius of the solid elements. Such a chamfering or rounding is advantageous to avoid that the solid elements accidentally leave the through holes or recesses while rotating.

According to a third aspect of the invention, there is provided a method for dosing and dispensing a predetermined number of solid elements using the device according to the first aspect of the invention or embodiments thereof. The method comprises:
rotating, from an initial position, the feeding unit relative the dispensing unit in said first direction a number of angular steps correlated to (or equal to) said predetermined number of solid elements, and
rotating the feeding unit relative the dispensing unit in said second direction back to said initial position.

The first rotating results in that said predetermined number of solid elements are fed into through holes or recesses, whereas the second rotating results in that said predetermined number of solid elements are dispensed through the dispensing opening.

The features of the embodiments described above are combinable in any practically realizable way to form embodiments having combinations of these features. Further, all features and advantages of embodiments described above with reference to the first aspect of the invention may be applied in corresponding embodiments of the second and third aspects of the invention and vice versa.

### BRIEF DESCRIPTION OF THE DRAWINGS

Above discussed and other aspects of the present invention will now be described in more detail using the appended drawings, which show presently preferred embodiments of the invention, wherein:

FIG. 1 shows an exploded view of parts of an embodiment of a device according to the first aspect of the invention;

FIG. 2 shows a cut-away view along a substantially vertical plane of the embodiment in FIG. 1 in assembled form;

FIG. 3 shows a detail view from FIG. 2;

FIG. 4 shows a cross section view of the embodiment in FIG. 1-4, the cross-section view being taken in a horizontal plane just above the first ratchet fingers of the feeding unit;

FIG. 5 shows a partial cross section view of the embodiment in FIG. 1-5 shown with solid elements in the container, the cross-section view being taken in a vertical plane;

FIG. 6 shows a side-top view of the embodiment in FIG. 1-5, the device being shown without the housing part;

FIG. 7 shows a cross section view of the embodiment in FIG. 1-6, the cross-section view being taken in a horizontal plane just below the first ratchet fingers of the feeding unit;

FIG. 8 shows an exploded view corresponding to FIG. 1 except that the release unit and the restricting member is also shown;

FIG. 9 shows a cross section view of the embodiment in FIG. 1-6, the cross-section view being taken in a horizontal plane through the dispensing unit, the release unit and the restricting member;

FIG. 10 shows side-top view of an embodiment of a device according to the first aspect of the invention, the device being provided with a stop member, and

FIG. 11 shows a cross section view of the embodiment in FIG. 10, the cross-section view being taken in a horizontal plane just above the first ratchet fingers of the feeding unit.

### DETAILED DESCRIPTION

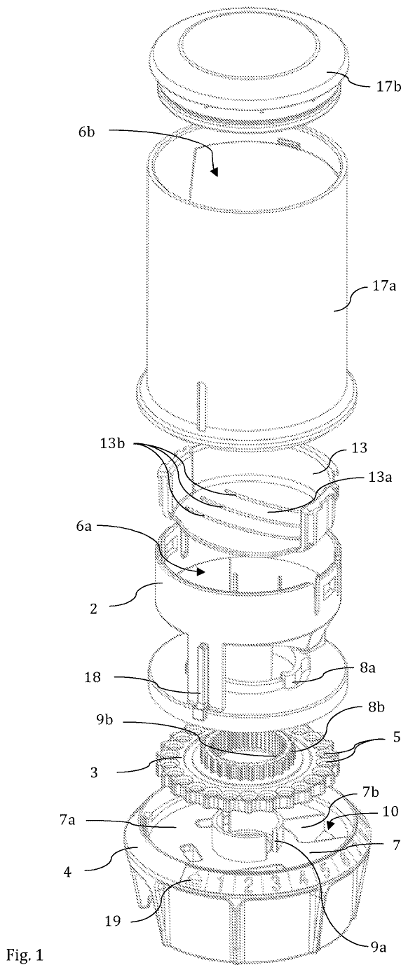

FIG. 1 shows an exploded view of parts of an embodiment of a device according to the first aspect of the invention. The device comprises a feeding unit 2, a dispensing unit 4 and a separating unit 3 sandwiched between the feeding unit and the dispensing unit. The feeding unit, separating unit and dispensing unit are arranged to rotate relative each other around a common axis of rotation.

The separating unit 3 is substantially circular in shape resembling a gear wheel. A plurality of through holes 5 are formed adjacent the periphery of the separating unit, i.e. in a circular formation. In other embodiments, the through holes may be replaced with recesses in the periphery of the separating unit forming chambers of corresponding size.

Each through hole has a size adapted to contain one tablet.

A compartment 6a, 6b for containing the tablets is formed by the feeding unit 2 and the housing parts 17a-b, where cylindrical housing part 17a is arranged coaxially around part of the feeding unit 2. The feeding unit is provided with a ridge portion 18 extending vertically (the vertical direction being defined by the common axis of rotation of the units 2-4), the ridge portion being received in a slit in housing part 17a when assembled such that the feeding unit 2 and housing part 17a rotate together.

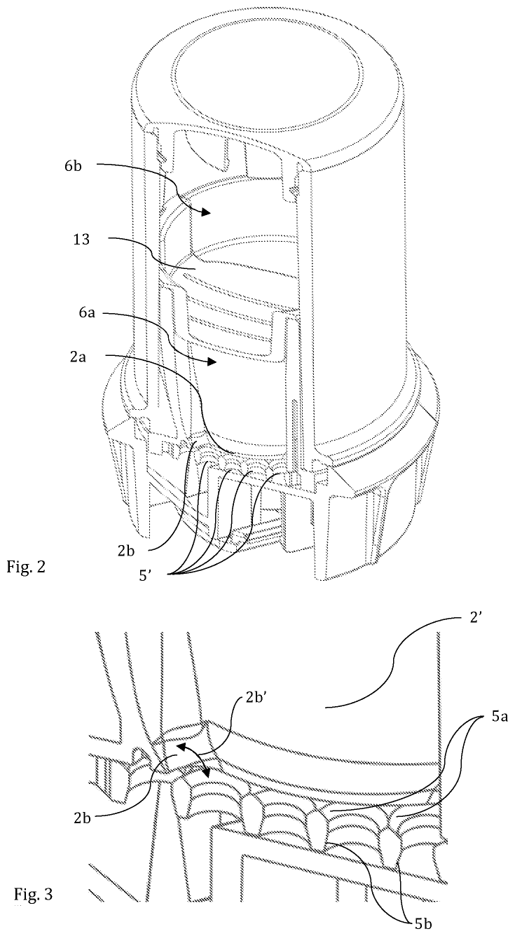

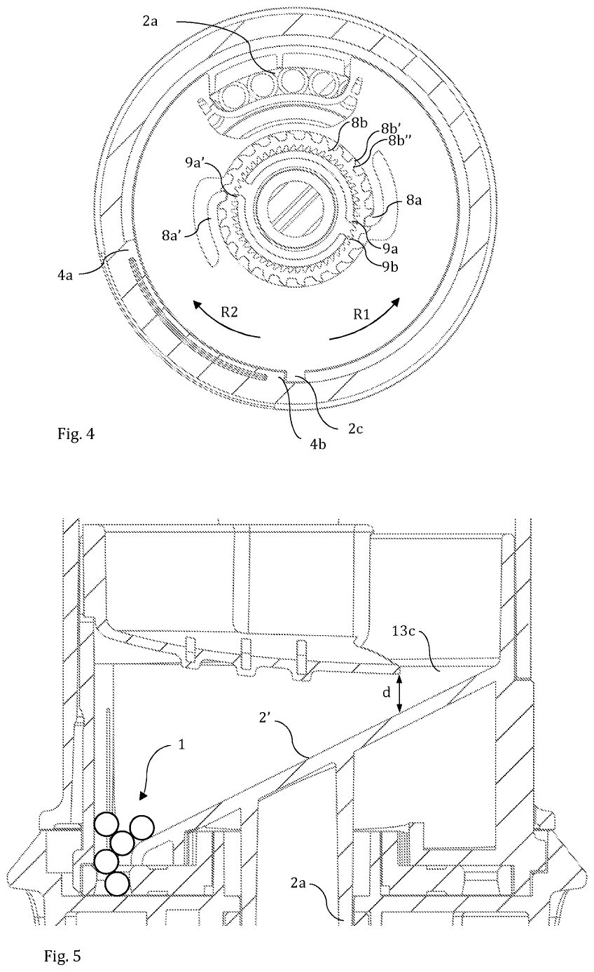

The feeding unit 2 is provided with a feeding opening 2a (see FIG. 4) towards the separating unit 3 to feed tablets from the compartment to each of four through holes (5′ for example, see FIG. 2) which are rotationally aligned with the feeding opening. The dispensing unit comprises a guiding surface 7 below the separating unit (assuming an upright position as shown in FIG. 1), the separating unit being arranged to rotate at a close distance from (or on) the guiding surface. The guiding surface comprises a retaining portion 7a arranged to form a lower wall portion for through holes of the separating unit which are rotationally aligned with the retaining portion, and a dispensing portion comprising a dispensing opening 7b arranged to allow solid elements contained in through holes of the separating unit which are rotationally aligned with the dispensing opening to be dispensed.

A first one-way rotation mechanism allowing rotation of the feeding unit relative the separating unit solely in a first direction of rotation and stepping means subdividing the rotation in the first direction into angular steps which correspond to an angular distance between one through hole are formed together as a ratchet mechanism comprising a first ratchet gear portion 8b of the separating unit and two first ratchet fingers 8a, 8a′ (see also FIG. 4) of the feeding unit engaging with each other. The first ratchet gear portion 8b is formed as an outer gear on an upwardly protruding circular flange portion of the separating unit at its inner periphery (see FIG. 1), i.e. a toothed profile at the periphery of the flange portion.

A second one-way rotation mechanism allowing rotational movement of the separating unit relative the dispensing unit solely in a second direction of rotation being opposite the first direction of rotation is formed as a ratchet mechanism comprising a second ratchet gear portion 9b of the separating unit and two second ratchet fingers 9a, 9a′ (see also FIG. 4) of the dispensing unit engaging with each other. The second ratchet gear portion is formed as an inner gear on the separating unit, i.e. a toothed profile at an inner periphery of the separating unit.

Further, as can be seen in FIG. 1, the dispensing unit further comprises a dispensing chamber 10 arranged in communication with the dispensing opening such that the solid elements are dispensed into the dispensing chamber.

The device further comprises a roof part 13 separating the compartment into an upper part 6b and a lower part 6a. As can be seen in FIG. 1, the roof covers part of the cross-section of the feeding unit such as to form at least one opening 13c (see FIG. 5) between the upper part 6b and the lower part 6a. A surface 13a inclines towards the lower part 6a of the compartment Three raised ribs 13b are arranged on the surface 13a, the ribs being arranged perpendicularly relative its direction of inclination.

FIG. 2 shows a cut-away view along a substantially vertical plane of the embodiment in FIG. 1 in assembled form. The cut-away view is shown to illustrate the feeding opening 2a and the four through holes 5′ rotationally aligned with the opening.

FIG. 3 shows a detail view from FIG. 2. As can be seen in FIG. 3, the feeding unit is provided with a feeding surface portion 2b adjacent the feeding opening 2a and facing in the first direction of rotation (R1, see FIG. 4), wherein the feeding surface portion inclines towards the dispensing opening at an angle 2b′ relative a plane having said common axis of rotation as a normal (a horizontal plane when the device is arranged vertically), the angle being 115 degrees. The feeding surface portion 2b has a width (radial extension defined by said common axis of rotation) being 1.2 times the diameter of the tablets.

Further, as can be seen in FIG. 3, the through holes 5′ are each provided with a chamfered/rounded inlet portion (5a for example) towards the feeding unit 2, wherein peripheries of the openings formed by adjacent inlet portions (5a for example) are in contact with each other. The chamfering/rounding of the inlet portions has a height being about 0.42 times a radius of the tablets. Further, the through holes are each provided with a chamfered/rounded outlet portion (5b for example) towards the dispensing unit. The chamfering/rounding of the outlet portions starts at a distance from the inlet being about 0.8 times a radius of the tablets.

FIG. 4 shows a cross section view of the embodiment in FIG. 1-4, the cross-section view being taken in a horizontal plane just above the first ratchet fingers of the feeding unit. In FIG. 4, the two first ratchet fingers 8a, 8a′ and the two second ratchet fingers 9a, 9a′ can be seen. The first ratchet gear 8b of the separating unit is formed with ratchet teeth having a leading side comprising first and second portions, wherein the first portion 8b′ is arranged radially inwards from the second portion 8b″, and wherein the first portion has a steeper inclination than the second portion.

The feeding unit is provided with a radially outwardly protruding rotation restricting member 2c being arranged to co-act with radially inwardly protruding stop portions 4a, 4b of dispensing unit 4. The feeding unit and the dispensing unit are rotatable relative each other between an initial position (2c at 4b) and an end position (2c at 4a) such that the dispensing opening and the feeding opening do not overlap.

FIG. 5 shows a partial cross section view of the embodiment in FIG. 1-5 shown with some tablets 1 in the container, thus also illustrating an embodiment of the arrangement according to the second aspect of the invention. The cross section is taken in a vertical plane. The feeding unit is provided with a tapering/funnel portion 2′ tapering towards the feeding opening, wherein the roof part 13, at the opening 13c, is arranged at a distance d from said tapering portion being 1.4 times the diameter of the tablets.

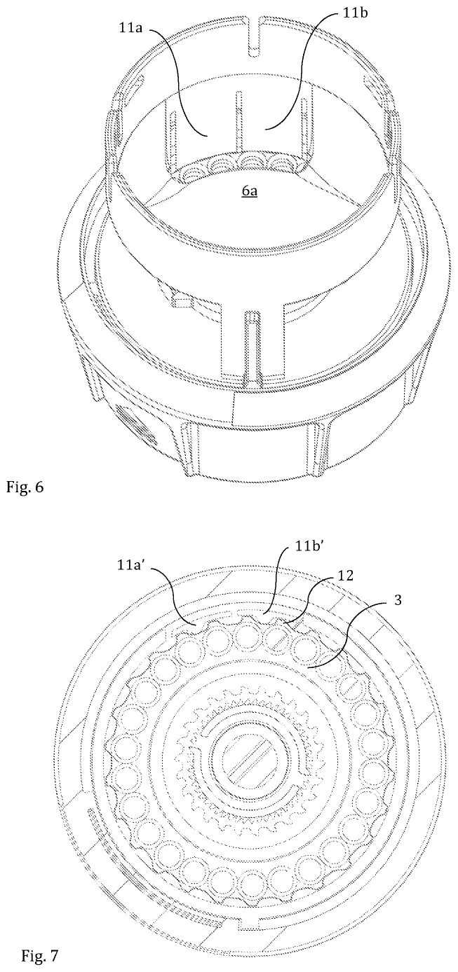

FIG. 6 shows a side-top view of the embodiment in FIG. 1-5, the device being shown without the housing part FIG. 7 shows a cross section view of the embodiment in FIG. 1-6, the cross-section view being taken in a horizontal plane just below the first ratchet fingers 8a, 8a′ of the feeding unit. The feeding device is provided with two parallel impacting elements 11a-b being integrally formed with the feeding device to form wall portions of the compartment 6a. The impacting elements are displaceable by means of each having a free end 11a′-b′ which is deflectable by means of contact engaging with a gear profile at the periphery of the separating unit 3. Thus, when the separating unit rotates, the free ends 11a′-b′ are displaced. The geometry of the compartment 6a thereby changes and the tablets therein are agitated/displaced which prevents the tablets from getting stuck. The impacting elements 11a-b agitate/displace the solid elements being in contact with (leaning/abutting against) the impacting elements which in turn agitate/displace neighboring solid elements in the compartment.

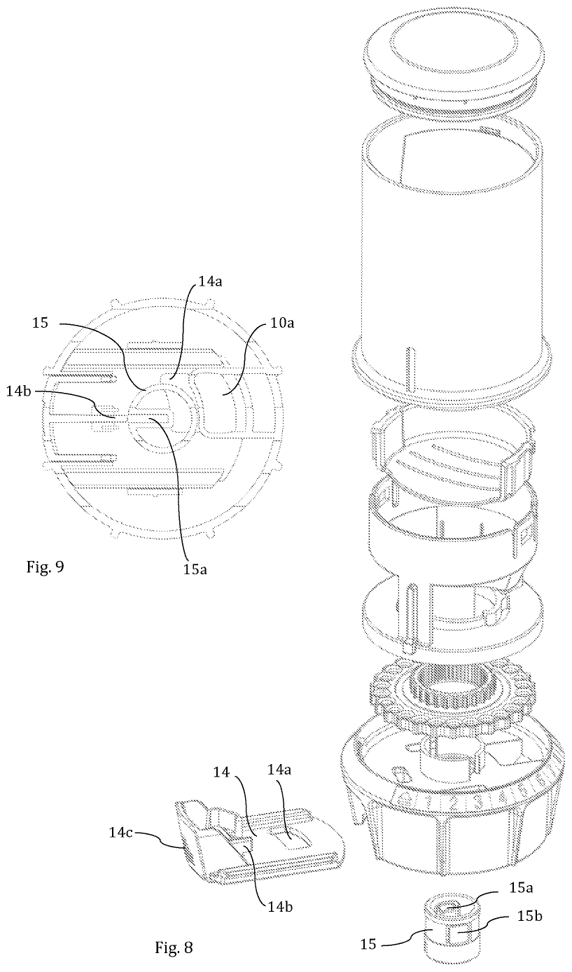

FIG. 8 shows an exploded view corresponding to FIG. 1 except that the release unit 14 and the restricting member 15 is also shown. FIG. 9 shows a cross section view of the embodiment in FIG. 1-6, the cross-section view being taken in a horizontal plane through the dispensing unit 4, the release unit 14 and the restricting member 15. As can be seen in FIG. 9, the chamber 10 of the dispensing unit 4 is provided with an outlet opening 10a, and the dispensing unit is provided with a release unit 14 being displaceable by a user of the device between a closed position and an open position, wherein in the closed position (shown in FIG. 9), the release unit blocks the outlet opening to prevent tablets from leaving the chamber, and wherein in said open position, an outlet (through hole) 14a of the release unit is aligned with the outlet opening to allow tablets to leave said chamber. The release unit is formed as a sliding element being resiliently loaded towards the closed position, said sliding element being provided with a push button portion 14c adapted to receive a pushing force from a user to overcome said resilient loading. A restricting member 15 is arranged to co-act with the feeding unit and the dispensing unit such as to allow the release unit to be displaced to said open position only at a predetermined relative position between the feeding unit and the dispensing unit. This functionality is achieved in that the restricting member rotates with the feeding unit 2, and is provided with a slot 15a, wherein when the feeding unit is rotationally aligned for ejecting the tablets, i.e. when it has been rotated back to the initial position (upwardly pointing arrow position), a protrusion 14b aligns with the slot 15a and can enter therein, which allows the sliding element to be pushed in only then.

The restricting member 15 has an additional function—to hold the feeding unit, separating unit and dispensing unit together. This is achieved by the restricting member engage with an axle portion 2a of the feeding unit (see FIG. 5) protruding (downwardly as seen in FIG. 8) axially through the separating unit 3 and partially through dispensing unit 4. The restricting member is provided with snap elements 15a, 15b engaging with through holes or recesses in the axle portion 2a when the restricting member is fitted in place to surround the (lower) end of the axle portion.

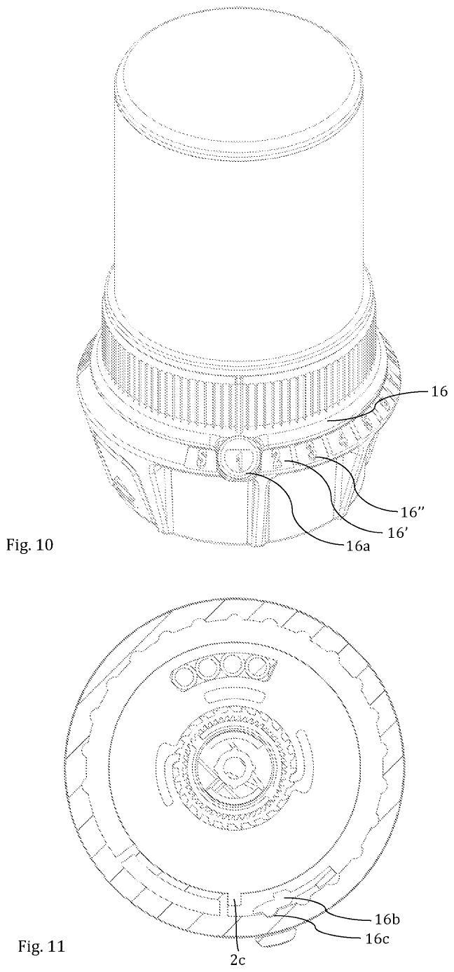

FIG. 10 shows side-top view of an embodiment of a device according to the first aspect of the invention, the device being provided with a stop member. FIG. 11 shows a cross section view of the embodiment in FIG. 10, the cross-section view being taken in a horizontal plane just above the first ratchet fingers of the feeding unit. The embodiment in FIG. 10-11 is identical to the embodiment in FIG. 1-9 except in that it further comprises a stop member being fixable at predetermined positions (16′, 16″ for example) around the periphery of the dispensing unit. The stop member is arranged to co-act with the separating unit and the dispensing unit to limit relative rotation therebetween to a number of angular steps corresponding to the predetermined position at which the stop member is fixed. The stop member comprises a sleeve portion 16 arranged coaxially with (and rotatable relative) the feeding unit, an indicating portion 16a being rotatable to any counter position (in FIG. 10-11 it is set to 1), and a limiting member 16b which extends vertically down into the interior of the dispensing unit. In FIG. 10, the stop member has been fixed at position 1, which is indicated by indicating portion 16a. The limiting member 16b will thereby prevent rotation of the feeding unit further than to counter position 1 by means of stopping the restricting member 2c of the feeding unit. As can be seen in FIG. 11, the limiting member 16b co-acts with recesses (16c for example) in the inner surface of the dispensing unit.

The device can be used as follows to dose and dispense tablets. Initially, the feeding unit 2 (and the housing parts 17a-b) is at the above-described initial position (which is also visually indicated in that the ridge 18 is at the start position 19, see FIG. 1). From the initial/start position, the feeding unit (and the housing parts 17a-b) are rotated relative the dispensing unit in the first direction a number of angular steps being equal to the desired number of tablets which is to be dispensed. The rotation of the feeding unit/housing parts in the first direction relative the dispensing unit is preferably achieved by means of the user holding/fixating the dispensing unit and rotating the feeding unit/housing parts in the first direction. Alternatively, the same relative rotation can be achieved by holding/fixating the feeding unit/housing parts and rotating the dispensing unit in the second direction of rotation (being opposite to the first direction). The number of angular steps/tablets is indicated by the ridge 18 on the numbered collar of the dispensing unit 4 (see FIG. 1). Assuming that the embodiment in FIG. 10-11 is used, the stop member is set to a number of steps being equal to the desired number of tablets, which prevents the feeding unit from being rotated further. The first rotating results in that the desired number of tablets are fed into respective through holes of the separating unit 3, but the tablets remain therein since the retaining portion of the dispensing unit 4 forms wall portions for the through holes which prevent the tablets from leaving.

In embodiments where the device does not comprise a numbered collar, the number of steps rotated can be manually counted by the user. Thus, the numbered collar is advantageous (but optional).

In embodiments where the device does not comprise a stop member, the user needs to rotate the feeding unit/housing parts more carefully to make sure that the number of steps is not exceeded. Thus, the stop member is advantageous (but optional).

After the above-described rotation in the first direction, the feeding unit (and the housing parts 17a-b) are rotated relative the dispensing unit in the second direction back to the initial position. The second rotation results in that the desired number of tablets are dispensed through the dispensing opening into the chamber 10. The rotation of the feeding unit/housing parts in the second direction relative the dispensing unit is preferably achieved by means of the user holding/fixating the dispensing unit and rotating the feeding unit/housing parts in the second direction. Alternatively, the same relative rotation can be achieved by holding/fixating the feeding unit/housing parts and rotating the dispensing unit in the first direction of rotation.

After the rotation in the second direction, the button portion 14c of the release unit 14 is pressed such that the outlet (through hole) 14a of the release unit aligns with the outlet opening 10a of the chamber 10 such that the (desired number of) tablets are ejected.

In embodiments where the device does not comprise a dispensing chamber 10 and a release unit 14, the solid elements are dispensed directly from the through holes of the separating unit during rotation, for example into the hand of the user, onto a table, into a drinking glass or the like. Thus, the dispensing chamber and the associated release unit are advantageous (but optional).

The description above and the appended drawings are to be considered as non-limiting examples of the invention. The person skilled in the art realizes that several changes and modifications may be made within the scope of the invention. For example, the through holes can be replaced with recesses (as explained above). Further the feeding opening can have an angular extension which corresponds to fewer or more through holes. Further, the ratchet mechanisms can be replaced with other types of one-way rotation mechanisms. Further, one or both of the housing portions can be integrally formed with the feeding unit. Further, the through holes can be adapted to each contain two or more tablets.

1. A device for dosing and dispensing solid elements, comprising:
a feeding unit;
a separating unit; and
a dispensing unit; wherein:
the feeding unit, separating unit and dispensing unit are arranged to rotate relative each other,
the separating unit comprises a plurality of recesses or through holes, each having a size such that the recess or through hole is capable of containing a predetermined number of solid elements,
a compartment for containing the solid elements is formed at least partly by the feeding unit, and the feeding unit comprises at least one feeding opening that opens towards the separating unit to feed solid elements from the compartment to one or more of the recesses or through holes that are rotationally aligned with the at least one feeding opening,
the dispensing unit comprises a guiding surface configured to co-act with the separating unit, the guiding surface comprising a retaining portion configured to form a wall portion for one or more recesses or through holes of the separating unit that are rotationally aligned with the retaining portion, and a dispensing portion comprising a dispensing opening configured to allow solid elements contained in one or more recesses or through holes of the separating unit to be dispensed,
the device comprises a first one-way rotation mechanism configured to allow rotational movement of the feeding unit relative the separating unit solely in a first direction of rotation,
the device comprises a second one-way rotation mechanism configured to allow rotational movement of the separating unit relative the dispensing unit solely in a second direction of rotation opposite the first direction of rotation, and
the first one-way rotation mechanism comprises, or is arranged to co-act with, stepping means subdividing the rotation in the first direction into angular steps that correspond to an angular distance between a predetermined number of the recesses or through holes.

2. The device according to claim 1, wherein the dispensing unit further comprises a dispensing chamber arranged in communication with the dispensing opening such that the solid elements are dispensable into the dispensing chamber.

3. The device according to claim 1, wherein the first one-way rotation mechanism and the stepping means are formed together as a ratchet mechanism comprising a first ratchet gear portion of the separating unit and at least one first ratchet finger of the feeding unit engaging with each other.

4. The device according to claim 1, wherein the second one-way rotation mechanism is formed as a ratchet mechanism comprising a second ratchet gear portion of the separating unit and at least one second ratchet finger of the dispensing unit engaging with each other.

5. The device according to claim 1, wherein the at least one feeding opening is provided as one feeding opening having an angular extension corresponding to an angular extension of the one or more of the recesses or through holes.

6. The device according to claim 5, wherein the angular extension of the one feeding opening corresponds an the angular extension of two to ten recesses or through holes.

7. The device according to claim 1, wherein the feeding unit comprises at least one impacting element arranged in the compartment or forming a wall portion of the compartment, the impacting element being at least partly displaceable such as to displace or agitate one or more solid element stored in the compartment, wherein the impacting element is displaceable by having a free end that is deflectable by contact with a gear portion of the separating unit.

8. The device according to claim 7, wherein the feeding unit comprises at least two impacting elements arranged in parallel.

9. The device according to claim 1, further comprising a roof portion separating the compartment into an upper part and a lower part, the roof portion covering part of a cross-section of the feeding unit so as to form at least one opening between the upper part and the lower part.

10. The device according to claim 9, wherein the roof portion comprises at least one surface inclining towards the lower part of the compartment.

11. The device according to claim 10, wherein one or more of the at least one surface comprises at least one raised rib or at least one slot, the at least one raised rib or at least one slot being arranged at an angle relative its direction of inclination.

12. The device according to claim 9, wherein the roof portion comprises a plurality of through holes.

13. The device according to claim 1, wherein:
the dispensing chamber of the dispensing unit comprises an outlet opening, and wherein the dispensing unit comprises a release unit that is displaceable by a user of the device between a closed position and an open position,
in the closed position, the release unit blocks the outlet opening to prevent solid elements from leaving the dispensing chamber, and
in the open position, an outlet of the release unit overlaps at least partly with the outlet opening to allow solid elements to leave the dispensing chamber.

14. The device according to claim 13, further comprising a restricting member arranged to co-act with the feeding unit and the dispensing unit by the restricting member being arranged to rotate with the feeding unit and being provided with a slot, and by the release unit being provided with a protrusion so as to allow the release unit to be displaced to the open position only at a predetermined relative position between the feeding unit and the dispensing unit when the protrusion is aligned with the slot to allow the protrusion to enter therein.

15. The device according to claim 1, further comprising:
a stop member that is fixable at predetermined positions around the periphery of the feeding unit and/or dispensing unit, the stop member comprising:
a sleeve portion arranged coaxially with and rotatable relative the feeding unit,
an indicating portion being arranged to indicate the predetermined position of the stop member, and
a limiting member that extends vertically down into the interior of the dispensing unit to act as a stop for a radially outwardly protruding rotation restricting member of the feeding unit, wherein:
relative rotation between the separating unit and the dispensing unit is limited to a number of angular steps corresponding to the predetermined position at which the stop member is fixed.

16. The device according to claim 1, further comprising:
at least one housing part, wherein:
the compartment is formed by the at least one housing part and the feeding unit.

17. The device according to claim 1, wherein the feeding unit comprises a feeding surface portion, the feeding surface portion being adjacent the feeding opening and facing in the first direction of rotation, wherein the feeding surface portion inclines towards the dispensing opening.

18. The device according to claim 17, wherein:
the feeding unit and the separating unit are arranged to rotate around a common axis of rotation, and
the feeding surface portion inclines at an angle relative to a plane having the common axis of rotation as a normal, the angle being within an interval 100-130 degrees.

19. The device according to claim 1, wherein:
at least two of the through holes or recesses are each provided with a chamfered or rounded inlet portion that opens towards the feeding unit, and
peripheries of the openings formed by adjacent inlet portions are in contact with each other.

20. The device according to claim 1, wherein at least two of the through holes or recesses each comprise a chamfered or rounded outlet portion that opens towards the dispensing unit.

21. The device according to claim 2, wherein:
the first ratchet gear of the separating unit comprises ratchet teeth having a leading side comprising first and second portions,
the first portion is arranged radially inwards from the second portion, and
the first portion has a steeper inclination than the second portion.

22. The device according to claim 1, wherein the guiding surface of the dispensing unit is arranged in abutment with the separating unit.

23. The device according to claim 1, wherein:
the separating unit has a substantially circular cross-section, and
the plurality of recesses or through holes are formed at or adjacent the periphery of the separating unit.

24. An arrangement comprising:
the device according to claim 1; and
a plurality of solid elements being tablets contained in the compartment of the device, the solid elements comprising medicine for the treatment or prevention of a disease and/or nutritional supplements.

25. An arrangement comprising:
the device according to claim 9; and
a plurality of solid elements being tablets contained in the compartment of the device, the solid elements comprising medicine for the treatment or prevention of a disease and/or nutritional supplements, wherein:
the feeding unit of the device comprises a tapering portion tapering towards the feeding opening,
the roof portion, at the opening, is arranged at a distance from the tapering portion, the distance being (n+d) times a size or diameter of one of the solid elements, where n is an integer ≥1, and d is within an interval from 0.1 to 0.7.

26. An arrangement comprising:
the device according to claim 12; and
a plurality of solid elements being tablets contained in the compartment of the device, the solid elements comprising medicine for the treatment or prevention of a disease and/or nutritional supplements, wherein:
the plurality of through holes each have a cross-section corresponding to (n+d) times a size or diameter of one of the solid elements, where n is an integer ≥1, and d is within an interval from 0.1 to 0.7.

27. An arrangement comprising:
the device according to claim 17; and
a plurality of solid elements being tablets contained in the compartment of the device, the solid elements comprising medicine for the treatment or prevention of a disease and/or nutritional supplements, wherein:
the feeding surface portion has a width being (n+d) times the size or diameter of one of the solid elements, where n is an integer ≥1, and d is within an interval from 0.1 to 0.7.

28. An arrangement comprising:
the device according to claim 19; and
a plurality of solid elements being tablets contained in the compartment of the device, the solid elements comprising medicine for the treatment or prevention of a disease and/or nutritional supplements, wherein:
the chamfering or rounding of the inlet portion has a height being within an interval of 0.2 to 0.7 times a radius of the solid elements.

29. An arrangement comprising:
the device according to claim 20; and
a plurality of solid elements being tablets contained in the compartment of the device, the solid elements comprising medicine for the treatment or prevention of a disease and/or nutritional supplements, wherein:
the chamfering or rounding of the outlet portion starts at a distance from a top of the through holes or recesses being within an interval of 0.5 to 1.5 times a radius of the solid elements.
